(19) United States
(12) Patent Application Publication
(10) Pub. No.: US 2015/0245777 A1
(43) Pub. Date: Sep. 3, 2015

(54) DETECTION OF EMOTIONAL STATES

(71) Applicant: BASIS SCIENCE, INC., San Francisco, CA (US)

(72) Inventors: Marco Kenneth Della Torre, Sydney (AU); Nathan Ronald Kowahl, San Francisco, CA (US); Jonathan K. Lee, San Carlos, CA (US); Jean Louise Rintoul, San Francisco, CA (US); Matthew Wayne Eckerle, Saint Louis, MO (US); Timothy Melano, San Francisco, CA (US)

(21) Appl. No.: 14/436,975

(22) PCT Filed: Oct. 21, 2013
(86) PCT No.: PCT/US2013/065965

§ 371 Date: Apr. 20, 2015

Related U.S. Application Data
(60) Provisional application No. 61/716,405, filed on Oct. 19, 2012.

(51) Int. Cl. A61B 5/026; A61B 5/0205; A61B 5/11; A61B 5/16

(52) U.S. Cl. CPC A61B 5/0261; A61B 5/165; A61B 5/0205; A61B 5/11

(57) ABSTRACT

A system and a method are disclosed for identifying and characterizing a stress state of a user based on features of blood flow identified from optical signals. One embodiment of a disclosed system (and method) includes an optical sensing system to detect features of blood flow and identify and characterize a stress state of a user based on those blood flow features. Light transmitted or reflected from tissue of the user is measured by an optical sensor. A processor analyzes the received optical signal to identify features of the blood flow. The stress state of the user is determined based on the identified features. The stress state is characterized according to a type of stress, a level of stress or both. Additionally stress events are identified.

### CROSS REFERENCE TO RELATED APPLICATIONS

This application claims the benefit of U.S. Provisional Application No. 61/716,405, filed Oct. 19, 2012, which is incorporated by reference in its entirety.

### BACKGROUND

1. Field of Art

The disclosure generally relates to the field of devices for measuring and characterizing stress.

2. Description of the Related Art

Stress is well known to be a major contributor to overall health. Convenient, continuous stress-monitoring devices that can be worn continuously and thus provide continual monitoring are currently lacking Current devices require a user to be tethered to a computer or monitoring device via wires needed for the monitoring apparatus to communicate with the computer or monitoring device. Measurement of stress levels is also available through the collection and analysis of bodily fluids such as sweat, saliva or blood.

### BRIEF DESCRIPTION OF DRAWINGS

The disclosed embodiments have advantages and features which will be more readily apparent from the detailed description, the appended claims, and the accompanying figures (or drawings). A brief introduction of the figures is below.

FIG. 1 illustrates one embodiment of a wearable device for measuring stress.

FIG. 2 illustrates one embodiment of the wearable device for measuring stress according to a second view.

FIG. 3 illustrates a top view and a bottom of one embodiment of the wearable device for measuring stress.

FIG. 4 illustrates a more detailed view of the wearable device comprising an optical sensing system.

FIG. 5 illustrates a system architecture of the wearable device according to one embodiment.

FIG. 6 illustrates a method for identifying and characterizing stress according to one embodiment.

FIG. 7 illustrates exemplary signal acquisition schema for identifying and characterizing stress.

FIG. 8 illustrates raw signal acquired from optical sensors according one embodiment.

FIG. 9 illustrates processed signal acquired from optical sensors according one embodiment.

FIG. 10 illustrates processed signal acquired from optical sensors according one embodiment.

FIG. 11 illustrates a method of identifying and characterizing stress including motion mitigation according to one embodiment.

FIG. 12 illustrates a method of identifying and characterizing stress including motion mitigation according to one embodiment.

FIG. 13 illustrates a method of identifying and characterizing stress including multiple sensors according to one embodiment.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

One embodiment of a disclosed device (and method) includes an optical sensing system to detect features of blood flow and identify and characterize a stress state of a user based on those blood flow features. Light transmitted or reflected from tissue of the user is measured by an optical sensor. A processor analyzes the received optical signal to identify features of the blood flow. The stress state of the user is determined based on the identified features. The stress state can be characterized according to a type of stress, a level of stress or both. Examples of the type of stress include relaxation, cognitive stress, emotional stress, physical, behavioral or general stress. The level of stress is identified and is determined independently of whether the type of stress is identified. Stress events can also be identified. Identifying stress events comprises detecting a time or time range during which a particular a type of stress or level of stress have been identified.

An individual's stress state can be ascertained by analyzing the individual's heart beats. The heart beats can be analyzed by analyzing the individual's blood flow. The amount of blood in any vessel or artery varies with changes in heart beats as blood is pumped through the body. The parameters of this variation, also referred to features of the blood flow, are related to stress the user is experiencing. Variation in blood flow and hence the blood flow features can be determined optically.

Light having a wavelength that is absorbed by blood is emitted onto the skin and into the underlying tissue. Blood traveling through the skin and tissue will absorb a portion of the emitted light. Some of the remaining light is reflected back and some of the remaining light continues through the tissue. If the body part onto which the light is emitted is small enough (like for example an ear lobe or fingertip), some of the light will be transmitted all the way through the body. Both the amounts of transmitted and reflected light are a function of how much light was emitted and how much light was absorbed by the blood. Thus measuring the transmitted or reflected light allows a determination the amount of blood present in the tissue at the time the light passed through. Sampling the transmitted and/or reflected light over a period of time provides information about the features of the blood flow.

The disclosed systems, devices (and methods) apply these principals to determining stress of a user wearing a wearable device. A processor on the wearable device or located remotely determines the stress state. Wearable technology enables people to interact with technology in a convenient and continuous manner, since it can be present on the body in the context of all lifestyle activities. An advantage of wearable technology extends to the device being able to measure the user and her surroundings continuously, as well as the ability to provide immediate information and feedback to the user at any time she has the device.

### Device

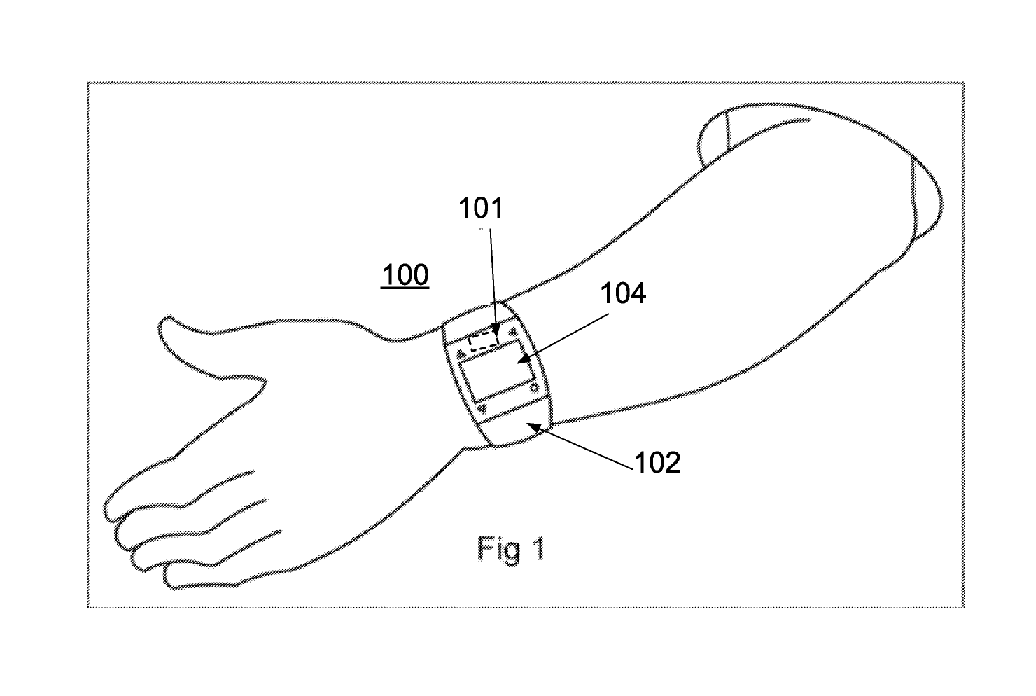

FIGS. 1 through 4 illustrate various views of a device for measuring stress according to the present disclosure. FIG. 1 illustrates an example of a wearable device 100 configured to be in close proximity to or in contact with a user. For example, the device 100 may be worn on a user's appendage or portion thereof, e.g., an arm or a wrist. A fastening system 102 fastens the device 100 to the user's appendage. The fastening system 102 may be removable, exchangeable, or customizable. Furthermore, although embodiments are described herein with respect to a wrist-worn device, other form factors or designed wear locations of the wearable device 100 may alternatively be used. For example, embodiments described herein may be implemented in arm-worn devices, chest-worn devices, head-worn devices, clip-on devices, and so forth. In one embodiment, the wearable device 100 is a physiological monitoring device for monitoring activities of its wearer and calculating various physiological and kinematic parameters, such as activity levels, caloric expenditure, step counts, heart-rate, and sleep patterns.

The wearable device 100 includes a display 104 and several user interaction points. The display 104 and user interaction points may be separate components of the device 100, or may be a single component. For example, the display 104 may be a touch-sensitive display configured to receive user touch inputs and display information to the user. The wearable device may also have a display element such as 104 without interaction points, or interaction points without a display element such as 104.

Generally, the device 100 comprises one or more processors 101 and an optical sensing system 203. Example processors 101 include the TIMSP430 from Texas Instruments and ARM Cortex-M class microcontrollers. The processor 101 is configured to use signals received from the optical sensing system 203 to determine an emotional state of the user at or around the time the signals were measured. The processor is further configured to determine a type, level and other parameters of the determined emotional state.

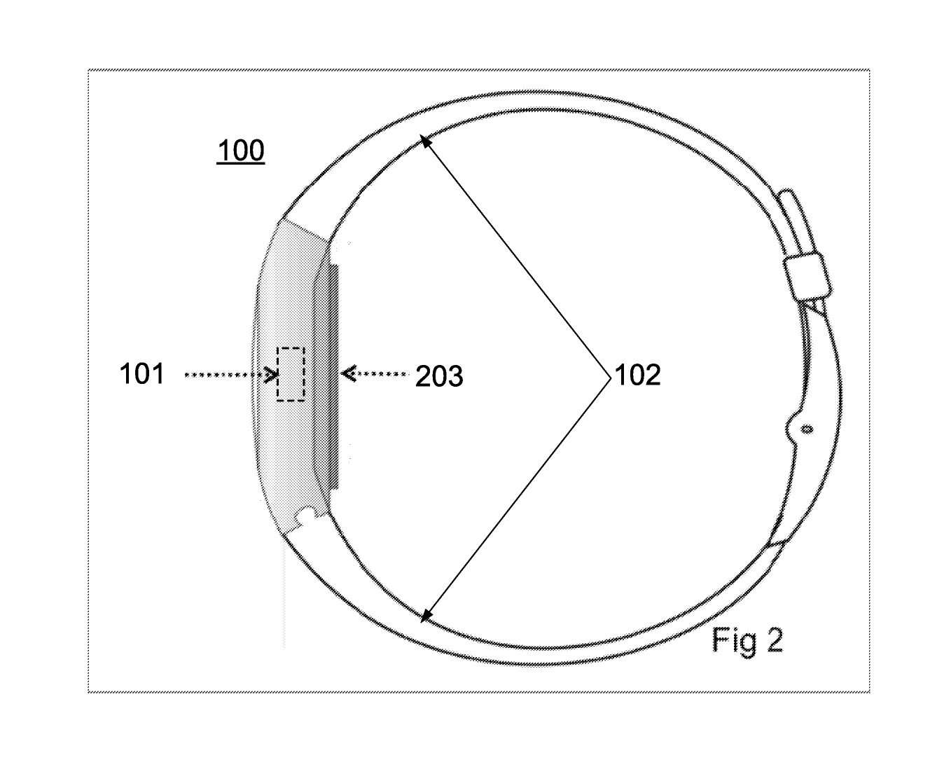

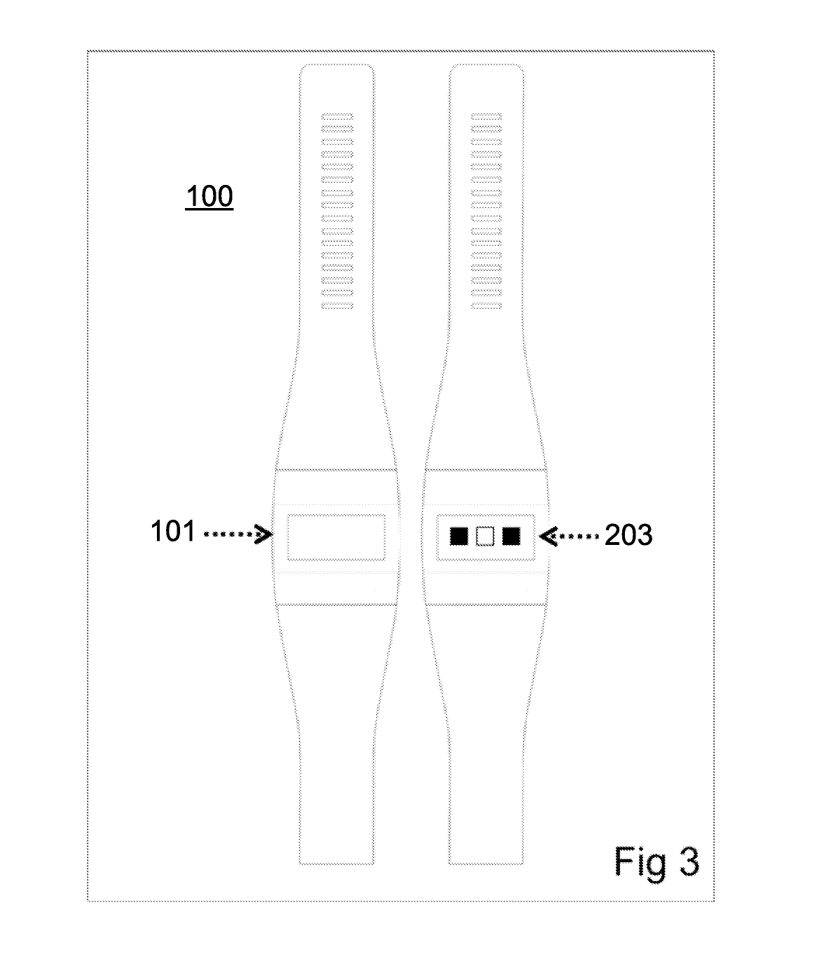

FIG. 2 illustrates a second view of the device 100 comprising the fastening system 102, processor 101 and optical sensing system 203. FIG. 3 illustrates a top view and a bottom view of the device 100. The processor 101 is inside the device and the optical sensing system 203 is visible on the bottom view of device 100. The side illustrated in the bottom view is the side in touch with the skin when device 100 is worn.

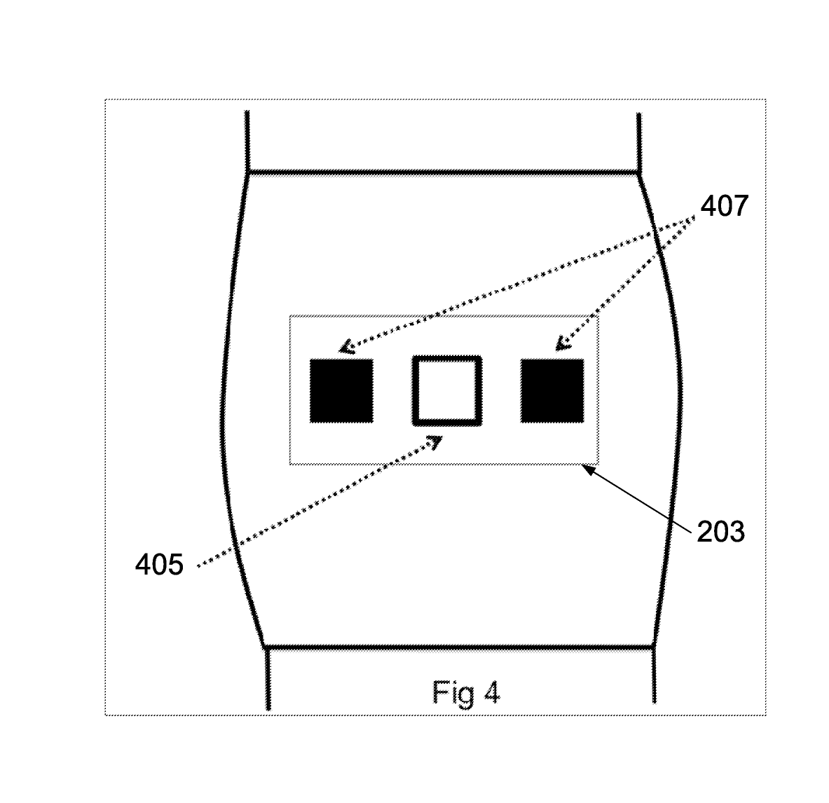

Turning now to FIG. 4, it illustrates the optical sensing system 203 according to one example embodiment. The system 203 comprises an optical sensor 405 and two optical emitters 407. Embodiments with one optical emitter 407, more than two optical emitters 407 or more than one optical sensor 405 are also possible. Signals from the optical sensor 405 are transmitted to the processor 101. Optical emitters 407 include light emitting diodes (LEDs) and lasers. In some embodiments light emitted from the optical emitters 407 is in the visible yellow and green ranges (500-600 nm). In other embodiments, light in the visible spectrum, such as blue and red, or the infrared spectrum may be used instead of or in addition to green light. Optical emitters 407 may emit light in other wavelengths in addition to those used for identifying blood flow features. For example, emitted light may be full spectrum white light.

In one embodiment, optical emitters 407 emit light at the same time, but in another embodiment the optical emitters 407 emit light in an alternating fashion. In another embodiment the optical emitters 407 may be set to emit light independently at some times and simultaneously at others. Optical sensor 405 detects light in the wavelengths of light emitted by the optical emitter 407. An example optical sensor 405 is Light To Voltage (LTV) sensor such as a Taos TSL13T or similar.

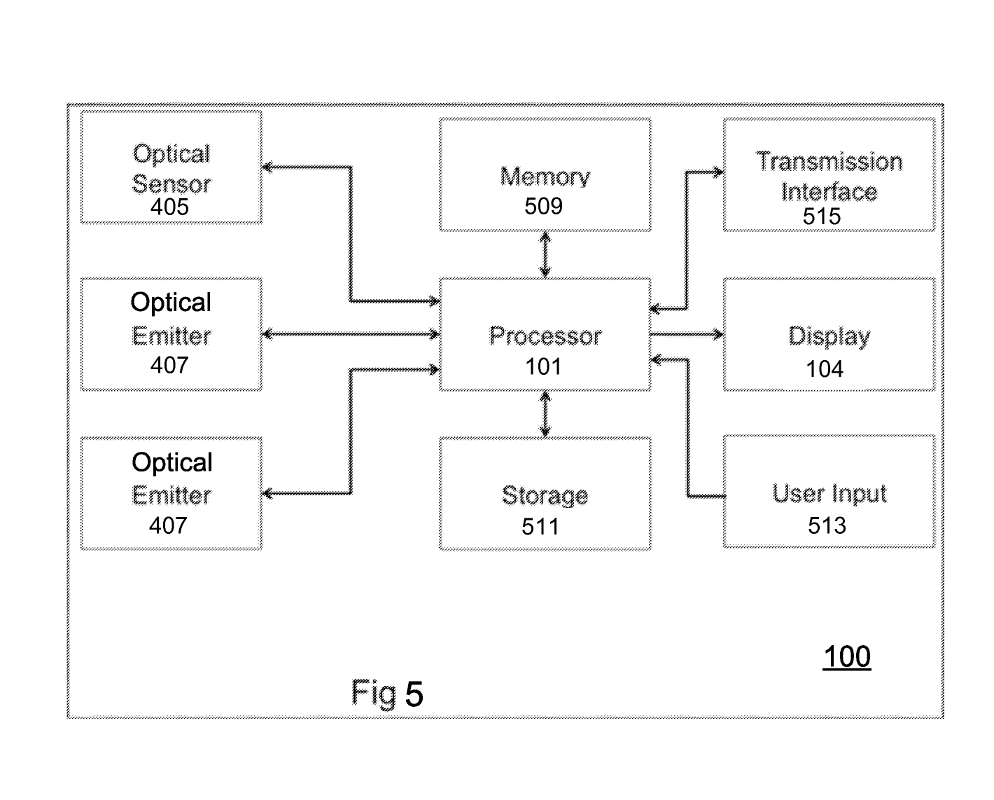

FIG. 5 illustrates a system architecture for the device 100. The device 100 comprises the processor 101, display 104, optical sensor 405, optical emitters 407, memory 509, storage 511, user input 513 and transmission interface 515. The memory 509 stores signals measured from the optical sensor 405 and parameters determined by the processor 101 from the measured signals, such as, for example, measurements of stress.

### Example Processing Configuration

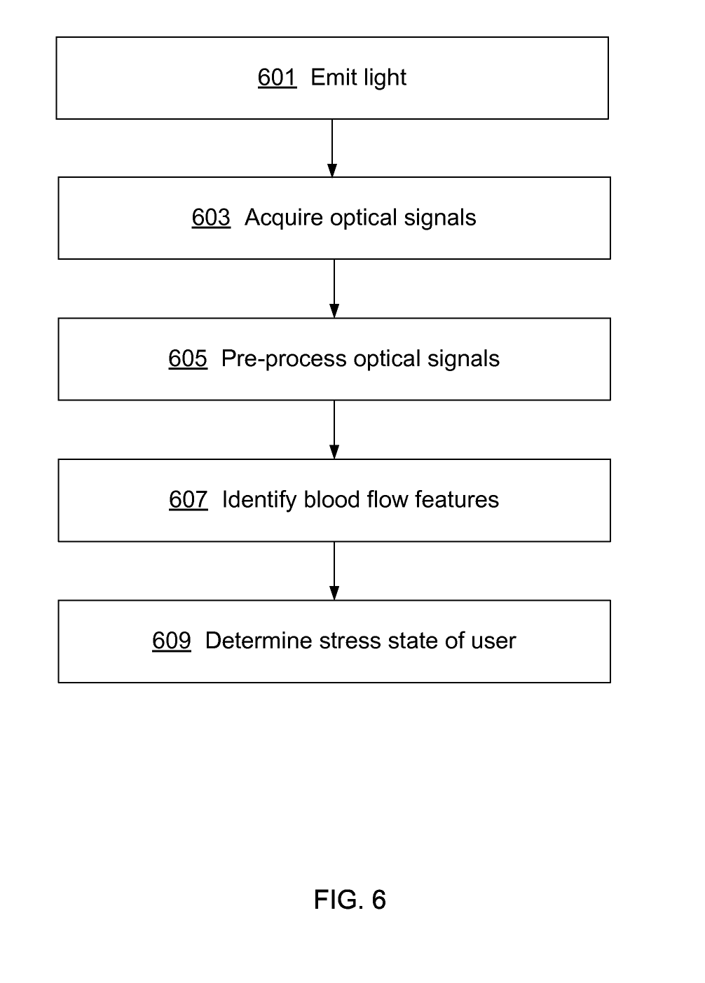

Referring now to FIGS. 6 through 13, illustrated is an example method for acquiring signals from the optical sensor 405 and determining parameters about the emotional state of the user at or around the time the signals were acquired. Specifically the method identifies and characterizes the user's stress state.

Referring first to FIG. 6, light is emitted 601 from the optical emitters 407. An optical signal is acquired 603 via the optical sensor 405. The emission of light and subsequent collection of signals can take place continuously while the device is worn by the user. Whether the device is being worn can be assessed through any means known in the art, including, but not limited to, use of a proximity sensor. Alternatively, the optical sensing system 203 is not continuously active. In some embodiments, the optical sensing system 203 generates data at intervals. The intervals may be regular or irregular. The processor 101 may activate the optical sensing system 203 in response to a stimulus such as the user waking up or the user can request a stress state determination at a particular time or on demand. In some embodiments, the optical sensing system 203 activates at regular intervals to generate data and additionally it activates in response to a stimulus.

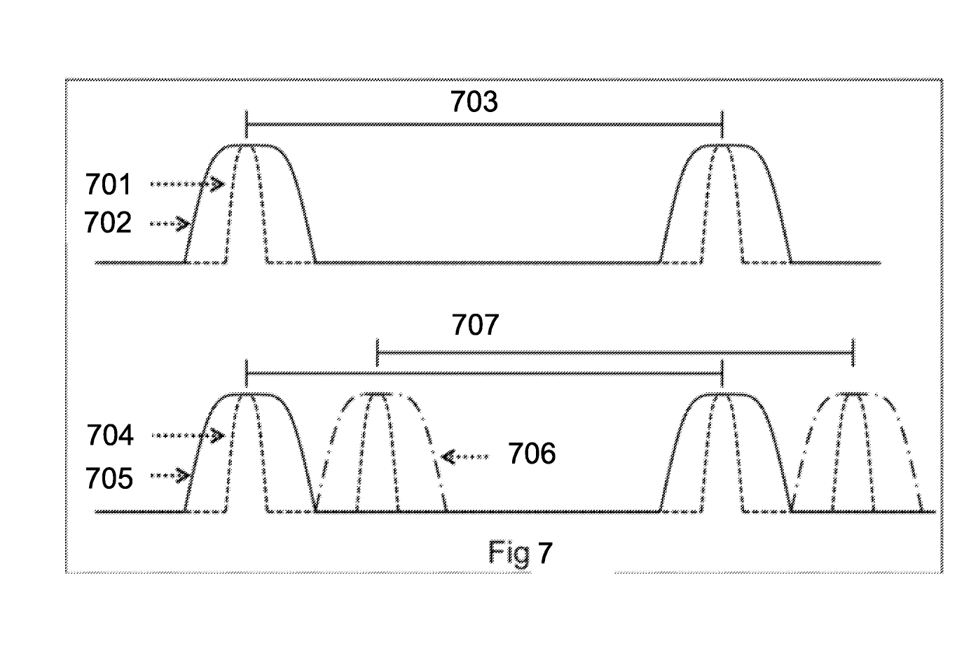

FIG. 7 illustrates example light emission and sensor sampling schemes. In a first embodiment, shown in the top graph, both optical emitters 407 emit light onto the skin at the same time. Line 701 illustrates activity of the optical sensor 405 and line 702 illustrates activity of the optical emitters 407. In each case the line illustrates when the respective component is on and off. Line 703 illustrates the sampling rate. In some embodiments, the sampling frequency is between 2 Hz-4096 Hz, 20 Hz-1024 Hz, 30 Hz-1000 Hz, 50 Hz-512 Hz, 64 Hz-512 Hz, 100 Hz-256 Hz or 128 Hz-200 Hz. In some embodiments the sampling frequency is 20, 30, 32, 50, 64, 100, 128, 200, 256, 500, 512, 1000 or 1024 Hz.

In a second embodiment illustrated the bottom graph of FIG. 7, the optical emitters 407 emit light onto the skin at different times in an alternating fashion. Line 704 illustrates activity of the optical sensor 405 and lines 705 and 706 each illustrate activity of one of the optical emitters 407. In each case the line illustrates when the respective component is on and off. Lines 707 illustrate the sampling rate. In some embodiments the sampling frequency for each of the optical emitters 407 is between 2 Hz-4096 Hz, 20 Hz-1024 Hz, 30 Hz-1000 Hz, 50 Hz-512 Hz, 64 Hz-512 Hz, 100 Hz-256 Hz or 128 Hz-200 Hz. In some embodiments the sampling frequency is 20, 30, 32, 50, 64, 100, 128, 200, 256, 500, 512, 1000 or 1024 Hz. In other embodiments, a combination of the two sampling schemes is utilized.

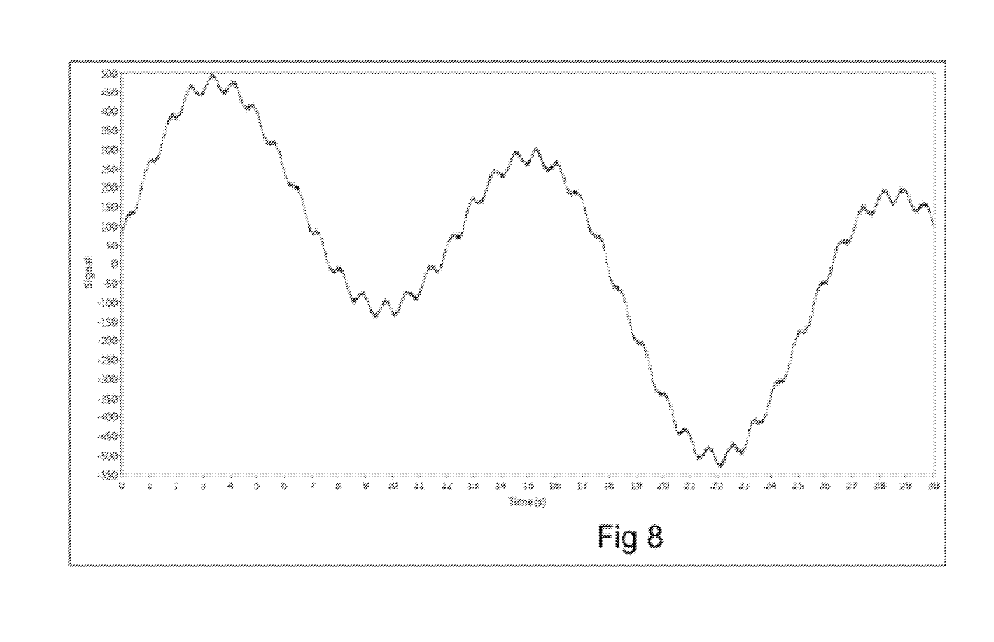

FIG. 8 illustrates an example of an acquired optical signal. The signal has several main constituents—a large, low frequency signal, a smaller, higher frequency signal and still smaller signal representing noise. The first two of these signals are analyzed because the levels of these two signals vary in response to the type or level of stress a user has experienced.

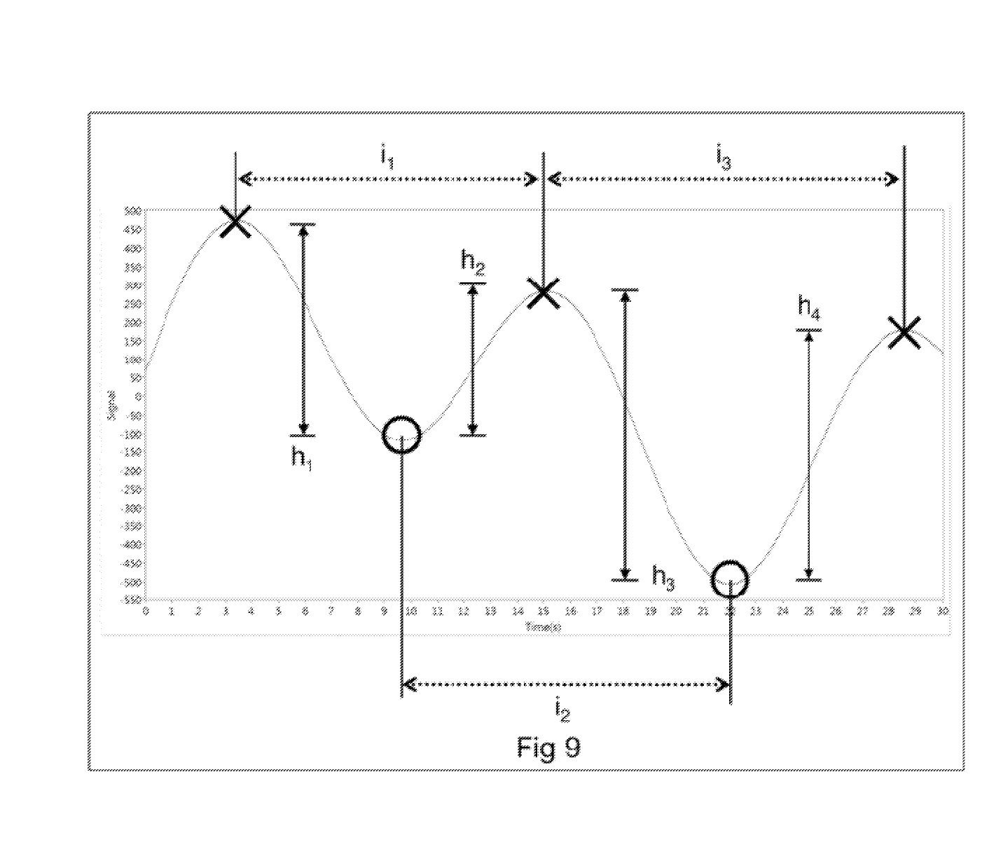

Referring back to FIG. 6, the acquired optical signals are pre-processed 605 in order to be able to ascertain various blood flow features from the signals. In one embodiment, pre-processing 605 comprises a filtering leaving the low frequency, large amplitude signal. Large, low frequency signals in the range 0.05-0.12 Hz are examined for frequency, magnitude, consistency of variance, relative power and other parameters. FIG. 9 illustrates determining some of these parameters. The intervals between subsequent peaks [i₁,i₃], and subsequent valleys [i₂], can be used to derive instantaneous and average frequency measures. The height from peak to valley [h₁,h₃], and valley to peak [h₂,h₄], may also be used as instantaneous or averaged magnitude measures. There are also techniques for measuring these features on an unfiltered signal. These features represent changes in the cardiovascular system that can be triggered by emotional changes in the body. For example, these may relate to a change in blood pressure, caused by an increase in stress.

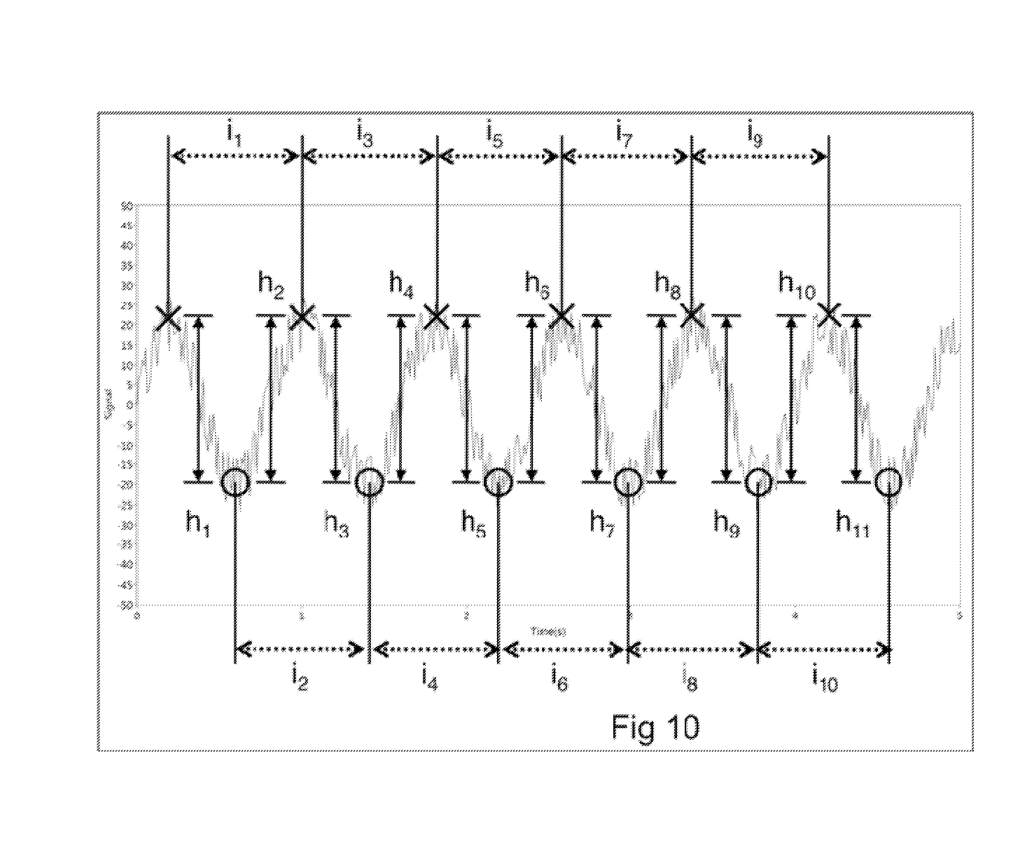

In another embodiment, pre-processing 605 comprises filtering leaving the higher frequency, smaller amplitude signal. Smaller, higher frequency signals in the range 0.5-2 Hz are examined for frequency, magnitude, consistency and other parameters. FIG. 10 illustrates determining some of these parameters. The intervals between subsequent peaks [i₁,i₃ . . . i₉], and subsequent valleys [i₂,i₄ . . . i₁₀], can be used to derive instantaneous and average frequency measures. The height from peak to valley [h₁,h₃. . . h₁₁], and valley to peak [h₂,h₄ . . . h₁₀], may also be used as instantaneous or averaged magnitude measures. There are also techniques for measuring these features on an unfiltered signal. These features relate to the heartbeat. The size and variance of these peaks may relate to a wide range of cardiovascular parameters that are modulated by emotional changes in the body. Since these peaks relate to blood flow features such as the peak absorption level observed by the sensor, changes in these signals can relate to cardiovascular changes due to emotional states.

Additionally, preprocessing can include assessing the quality and/or quantity of the collected data. In some embodiments this comprises determining an amount of time or a number of heart beats between interruptions to the data. Data collection can be interrupted by interference in the optical signals due to motion, for example.

Table 1 lists exemplary blood flow features that can be used to determine a user's stress state. In some embodiments, these blood flow features are determined from the optical signals. Table 1 includes four categories of blood flow features that can be determined from the optical signal. The features in two of these categories, 2 and 4, are features that are not available via electrocardiogram (ECG), a more conventional method used to assess stress states in individuals. These signals relate to peripheral vascular activity that can be indicative of the impacts of emotional states on the body. The features in category 1 can be determined via ECG and thus in other embodiments, if blood flow features from category 1 are used to determine stress states, the input data can be from ECG in addition to or in place of optical data.

TABLE 1
Category 1: Statistical measures

| Variable | Description |
|---|---|
| Instantaneous Heart Beat Interval | A single inter-beat (RR) interval within the |
| | measurement time window. i₁ and i₃ of FIG. 9 |
| | illustrate this measure. |
| Heart Beat Interval Mean & Std | Mean and standard deviation of all inter-beat (RR) |
| | intervals within the measurement time window. |
| Heart rate (HR) Mean & Std | Mean and standard deviation of all heart rates within |
| | the measurement time window. |
| HR Mean & Std Norm | Mean and standard deviation of all heart rates within |
| | the measurement time window with a baseline heart |
| | rate subtracted to normalize between subjects. |
| RMSSD | Square root of the mean of the sum of squares of |
| | differences between adjacent RR intervals. |
| NN50 count | Number of RR intervals exceeding 50 ms within the |
| | measurement time window. |
| pNN50 | NN50 count divided by the total number of all RR |
| | intervals in the measurement time window. |
| NN20 count | Number of RR intervals exceeding 20 ms within the |
| | measurement time window. |
| pNN20 | NN20 count divided by the total number of all RR |
| | intervals in the measurement time window. |
| RR Range | Maximum RR interval-Minimum RR interval |
| RR spectral purity | Magnitude of the peak frequency of the RR interval |
| | pattern, divided by some or all of the magnitudes in |
| | adjacent frequencies |

Category 2: Time Domain Peak Morphology

| Variable | Description |
|---|---|
| Median Heart Rate Frequency | The median peak to valley and valley to peak height. |
| Peak height | May alternatively use valley to peak height, or both. |
| | Peaks in the 0.3 Hz to 3.5 Hz range only are considered, |
| | specifically those corresponding to frequencies near the |
| | heart rate. |

Category 3: Frequency Domain Analysis

| Variable | Description |
|---|---|
| Peak Frequency | Peak frequencies of the power spectral density (PSD) |
| | estimate for the VLF, LF, and HF frequency bands. |
| | [3 Variables] |
| Very Low Frequency (VLF) | Power from (0-0.04] Hz. |
| Low Frequency (LF) | Power from [0.04-0.15) Hz. |
| High Frequency (HF) | Power from [0.15-0.4) Hz |
| LF Normalized | LF power in normalized units: LF/(Total Power − VLF)*100. |
| HF Normalized | HF power in normalized units: HF/(Total Power − VLF)*100. |
| LF/HF Ratio | LF/HF |
| Triangular Index | Starting with a histogram of RR intervals, group |
| | intervals into discrete ranges [50-1000 ms each] and |
| | divide the number of entries in the modal range [i.e. |
| | the range with the most number of entries] by the sum |
| | of the number of entries in all the other ranges. |

Category 4: Time Domain Analysis

| Variables | Descriptions |
|---|---|
| Low Frequency Wave | Peak detect over filtered PPG waveform to extract |
| | mean period of wave in the 0.05 Hz-0.12 Hz frequency |
| | range. |
| Low Frequency Wave Correlation | Pearson correlation with 0.05 Hz-0.12 Hz wave |
| | frequency sinusoid. This is the maximum or average |
| | value. |
| Low Frequency Wave Amplitude | Mean peak to peak amplitude of the low-frequency |
| | [0.05-0.12] Hz wave over the epoch. |

The identified blood flow features are used to determine 607 information about the user's stress status. In a first embodiment, a pre-trained algorithm is used to convert the observed feature levels to an observed stress type, level or other descriptor. The algorithm is trained in advance using data collected from subjects experiencing the types, levels and other categories of stress to be detected. Alternatively, thresholds for various blood flow features and combinations of each feature's level are used. For example, if a certain proportion of features are beyond a threshold set to indicate stress, the data analyzed could then be considered to represent an elevated stress state.

Each of the above-identified methods for determining 607 the user's stress status can be normalized for individual users. In one example, resting heart rate is determined for a user after the device is purchased and activated. That resting heart rate is used to normalize the data for the user. Normalizing the process for the user is described in further detail in Example 2.

In some embodiments, processor 101 determines a quality or quantity of the collected optical data and determines the user's stress state only when the quality and/or quantity of data exceeds a threshold. The thresholds for sufficient quantity or quality of data can be different based on the context of the user as the data is collected. If a user is moving vigorously, fewer of the user's heartbeats may be collected due to interference in the optical signal from motion. Ideally, in order to determine the user's stress state, the processor 101 uses data from the majority of the user's heartbeats in a given period of time (for example 3 or so minutes). However if the processor 101 determines that the user is moving, the processor 101 applies a lower threshold as to what is sufficient data to determine the user's stress state. While the assessment of the stress state may not be as accurate as when the higher threshold is applied, it is more useful to the user to have an assessment of stress state as opposed to no assessment.

The determined stress state is stored and is displayed to a user via the display 104 in response to a query received via the user interaction points on the face of the device 100. Additionally or alternatively, the stress status is displayed on the display 104 automatically. The status may be displayed automatically if the level of stress determined by the processor 101 exceeds a threshold. Providing the information to the user automatically is useful to alert the user that she is experiencing a high amount of stress. The processor 101 may also provide for display information to assist the user in reducing stress.

In some embodiments, alternative biological parameters are used in combination with the blood flow features in the determination of an individual's stress state. Table 2 identifies exemplary parameters that can also be used.

TABLE 2
Alternative Biological Signals

| Variables | Descriptions |
|---|---|
| Skin | Skin temperature gradient, minimum, maximum, |
| Temperature | average, range, or standard deviation over time. The |
| | presence of changes may also be used to signify a skin |
| | temperature event, which can then be used as a |
| | variable. |
| Temperature | Difference between skin and ambient temperature, be |
| difference | it the minimum, maximum, average, range, or standard |
| | deviation over time. The presence of changes may also |
| | be used to signify a temperature difference event, |
| | which can then be used as a variable. |
| Blood Pressure | Blood pressure minimum, maximum, average, range |
| | or standard deviation over time. May be estimated from |
| | the optical data via low-frequency signal oscillations |
| | or pulse speed measurements. The presence of changes |
| | may also be used to signify a blood pressure event, |
| | which can then be used as a variable. |
| Electrodermal | Skin conductance changes over a time window, |
| Activity | including gradient, range, maximum or standard |
| (EDA) | deviation. Patterns such as the ration of the time to rise |
| | versus the time to fall. |

In one embodiment data is collected from users experiencing various stress types, levels of stress and stress events, a data set for each stress type to be detected can be prepared. These data sets are then used to train a classifier such as a Support Vector Machine (SVM), Random Forest or other machine learning techniques. The classifier can be trained with all or some of the features of Tables 1 and 2. When data is presented to the system from a user, it can be decomposed into the features used to train the classifier which in turn allow for classification of the stress state of the user.

In an alternative embodiment, determination of a user's stress state may occur at a remote processor instead of on device 100. In such an embodiment, the remote processor implements the functionality of processor 101. The process described in FIG. 6 for determining a user's stress state can be apportioned between processor 101 and the remote processor in various ways. In some embodiments, device 100 transmits raw signal acquired from the optical sensor 407 to the remote processor and all processing is accomplished on the remote processor. In other embodiments, identified features of blood flow are transmitted to the remote processor. Transfer of the process between the device 100 and remote processor can occur at any step in between as well.

The received stress data and determined stress states can be stored remotely regardless of where the stress states were determined. For example, the information can be stored on a remote server so that the user can access the information via multiple devices such as a laptop computer, tablet computer, smartphone and the like. Guidance for the user on managing her stress states can then be provided to the user on these other devices. This is useful as the display 104 on device 100 has limited space for displaying information.

Additionally, the remote server may store various other information about the user such as a calendar application. In such an embodiment, the remote server may match the time stamps from the collected data and determined stress state to calendar entries for the user and display the user's stress states during a given time period next to the calendar from that time period. The remote processor may also instruct processor 101 to activate the optical sensing system 203 during times when the calendar application indicates there is an appointment. This is more beneficial to a user than having the stress state only determined at predetermined intervals such as every half hour.

### EXAMPLE 1

In this example the features of Category 1 in Table 1 were used. The smaller feature set was found to be predictive and is thus useful as it reduces processing. When the method is implemented on a wearable device, it beneficially allows for longer data collection on the limited storage in the wearable device.

In order to develop the algorithm, studies were performed with a cohort of subjects varying in age, gender, background and physical dimensions. During these studies, subjects were subjected to various situations and stimuli designed to elicit particular emotional states. The study was performed with three phases:
1. Phase 1—Subjects were allowed to relax in a calm setting, with minimal ambient noise and in comfortable seating. They were instructed to enjoy a calm, relaxing time while staying awake.
2. Phase 2—Subjects engaged in a series of cognitive exercises under time pressure to induce a state of cognitive stress.
3. Phase 3—Phase 1 was repeated to induce a state of calm.

The timing of each phase was recorded so that the biometric data from each phase could be identified and labeled with the specific emotional state of the subject. The data recorded in this study includes data from a wearable device such as that described above, as well as other devices.

Signals that can be used in this experiment include:
1. Blood flow, via an optical sensing system such as that described above
2. Motion, via an accelerometer, pressure sensor or similar sensing modality
3. Skin temperature
4. Core temperature
5. Ambient temperature
6. Galvanic skin response
7. Cortisol levels
8. ECG
9. Ambient humidity
10. Electro-dermal activity (EDA) from a second site (if a wrist-worn device was used, a finger may be used to collect EDA).
   Other signals such as blood pressure (11) and respiration rate (12) (as determined from heart beat variance or otherwise) could also be used.

Some of these signals are sensed continuously, at rates of 1 Hz or faster (1,2,3,4,5,6,8,10) and some are sampled at specific times before, during and after the study (7,9,11).

In the experiment users answered surveys after each phase of the study, indicating their level of different types of emotions including relaxation, cognitive stress, frustration, anxiety and arousal. An initial study including 30 subjects was performed, but subsequently extended with more subjects and a wider range of demographics. In one instance, the signals recorded were: heart beats (via an optical sensor), motion (via an accelerometer), ECG and cortisol levels.

In subsequent studies, different stimuli were used and data sets processed in the manner outlined below. In one such subsequent study, the signals recorded were: heart beats (via an optical sensor), motion (via an accelerometer), skin temperature, ambient temperature, galvanic skin response, ECG, and cortisol levels.

Data from such a study was used to train a detection algorithm. This algorithm was then used to classify data from other subjects, recorded from their normal lives, into one of the states the algorithm aimed to identify. In one experiment, data from each subject state was labeled (for example in the study described above these labels could reflect a calm set and a stressed set). Data for a particular class from all subjects was combined into a large pool to represent the biological response to each emotional state.

For each subject in the study, the data collected from each class was segmented into time windows. In one embodiment this window was 3 minutes. Overlapping segments were used. For example, the first three minutes of the calm phase could be a time window, as could the three minutes between the first and fourth minute. In this way, the second time window overlaps the first by one minute. If an overlapping system is used, the overlap can be as much as one second less than the window size, or as small as one second.

The data from each time window was labeled with the class it was taken from and decomposed into features such as those in Tables 1 and 2. The duration of the time window for each individual feature may be different.

In the process of training, or by explicitly analyzing each feature's variance in the two states, it was possible to determine which features are most able to distinguish between the given classes to be detected. All features can be used, however it may also be advantageous only to use a subset that most effectively distinguish the classes or a subset that is most convenient to measure subsequently from device wearers. One embodiment of a classifier architecture that facilitates this feature selection was a Random Forest classifier. This classifier not only trains a model to separate the classes under consideration, but can also produce metrics as to which features are most important in separating classes. If more than 2 emotional states are to be detected, different features may be most powerful at separating different combinations of classes.

Other classifiers may be used, such as, but not limited to, Linear Discriminant, Support Vector Machine, Linear Regression or Neural Network. A combination of classifiers may be optimal, since different class combinations may be more optimally separated by different classifier architectures. Different classifier architectures may be best at separating classes with different feature sets. For example, even when classifying the same classes, the features that are optimal for a Random Forest classifier may not be the same as features that perform best if a Linear Discriminant is used.

Once a classifier has been trained, it is possible to take data measured from a wearable device such as that described above and have the classifier output a score representing the likelihood that the data belongs in one of the classes available for classification. In the same way that time windows were created and decomposed into features to train the classifier, data of the same duration time window is collected and decomposed into features. The features are then used to evaluate the emotional state of the wearer at that time.

The time window that is used to train the classifier may be different to that used to evaluate a stress level using that classifier. For example, a classifier was trained using 3 minute time windows, but was used to evaluate recordings of just 1 minute duration.

The likelihood may be represented as a level, for example the degree to which the user is in one state versus another, or as a means to detect events over time. Events may be detected by thresholds that identify a change in likelihood over a period of time. For example, if a stress likelihood score from a range of 0 to 1 were to increase by more than 0.25 over 5 minutes, this could be classified as a stress event.

It should be noted that, where feature data can be sourced from multiple sensors, a different sensor may be used to train the algorithm than to operate it as a detector. For example, one feature used was Heart Rate Mean & Std, however the algorithm was trained using heart beat data from the ECG and then used as a detector via heart beat information sourced from a wearable optical sensor.

### EXAMPLE 2

### Normalization

In order to correct for inter-subject differences, data may be used for calibration or normalization. Normalization method parameters may include biasing terms (addition and subtraction), scaling terms (multiplication and division), or other non-linear processing parameters such as raising variables to a given power, and remapping the feature space via logarithmic, exponential or logistical transforms. Normalization parameters may be derived from scientific literature, or dynamically from the data itself using statistical or unsupervised learning techniques.

For example, one feature that may be important in assessing the presence of an emotional arousal state is an increase in heart rate magnitude. Since different subjects may naturally have different heart rate magnitude levels when calm or aroused, it may be necessary to normalize a subject's observed heart rate magnitude. In one embodiment, the user's data is recorded during a 24 hour period and this data is used to generate a biasing and scaling term. A median and standard deviation of heart rate magnitude during relatively inactive periods could be used to normalize for an individual. By subtracting the median from the subject's observed heart rate magnitude values and dividing by the standard deviation, the observed measures of heart rate magnitude may be normalized to similar levels for all subjects.

In another embodiment, heart rate is scaled against an estimate of the user's maximum heart rate. One estimate for maximum heart rate is 220−(user's age).

Another feature that may be important in assessing the presence of an emotional arousal state is a decrease in heart rate variance. Since different subjects may naturally have different heart rate variance levels when calm or aroused, it is possible to normalize by subtracting a subject's baseline heart rate variance level. In one embodiment, the user's data is recorded during sleep and this data is used to generate a baseline. A median of inter-beat intervals during sleep could be used by subtracting this value from observed measures of inter-beat intervals during the day.

In another embodiment, the biasing and scaling terms may be derived via an unsupervised learning method (such as k-means clustering with k=2). In this embodiment the observed heart rate magnitude will be biased by the average of the centroids given by the unsupervised learning method, and scaled by the magnitude of the centroid distances from each other.

In subsequent use of the trained classifier, a users' data may again be normalized before being processed by a classifier to correct for their personal baseline, maximum, or range.

### EXAMPLE 3

### Weighted Training Example

Different subjects respond to stimuli in the data collection studies in different ways. While some stimuli will make one subject emotionally aroused, it may not have the same impact on the emotional state of another subject. In order to correct for this inter-subject variance, it is possible to use objective or subjective measures of actual response to weight the impact of each subject's data for a given class in the development of a classifier for that class.

For example, in the study described above, the survey results after each phase could be used to weight the time windows during training The responses to a question asking the level of relaxation could be used as follows:
For the calm phase, survey responses with higher relaxation scores would be weighted more heavily and contribute more to the classifier training
For the stress phase, survey responses with lower relaxation scores would be weighted more heavily and contribute more to the classifier training

The same weighted training scheme can be used with cortisol measurements taken at transitions in the study (for example, at the start of each phase). Individual measurements, or an overall measurement for the study can be used to weight the contribution of data from any one subject. Since cortisol is a hormone released in response to stress, and since hormone release can take much longer than the duration of a stimulus, measurement over time, after the study is complete, can give more insight into how stressful a study was for a subject.

### EXAMPLE 4

### Adaptive Operation Example

Once a classifier has been trained, its operation can be updated in an ongoing basis to better match a single user's biosignals over time. For example, as baseline measures of cardiac function such as resting heart rate and heart rate variance change, the normalization process that is used to generate more consistent features can also evolve. In this way, the algorithm continues to adapt to a user over time and, thereby, maintains the accuracy of its detection of emotional states over time.

In one embodiment, the system measures the resting heart rate by estimating it during and around times of sleep. This measurement is then used to normalize a feature based on heart rate, by subtracting the most recent resting heart rate measurement. In this way, while a user's heart rate may change, the system is constantly updating their resting heart rate value and normalizing their heart rate based feature with that most recent measurement of resting heart rate. Not only does this normalize across different subjects, but also across time for a single subject.

### EXAMPLE 5

### Motion Mitigation Example

The optical signals related to blood flow information include noise introduced by motion of the wearer. In order to mitigate such noise interfering with the detection of heart beats and other information, motion sensing may be used. Example motion sensors include an accelerometer, gyroscope, pressure sensor, compass and magnetometer.

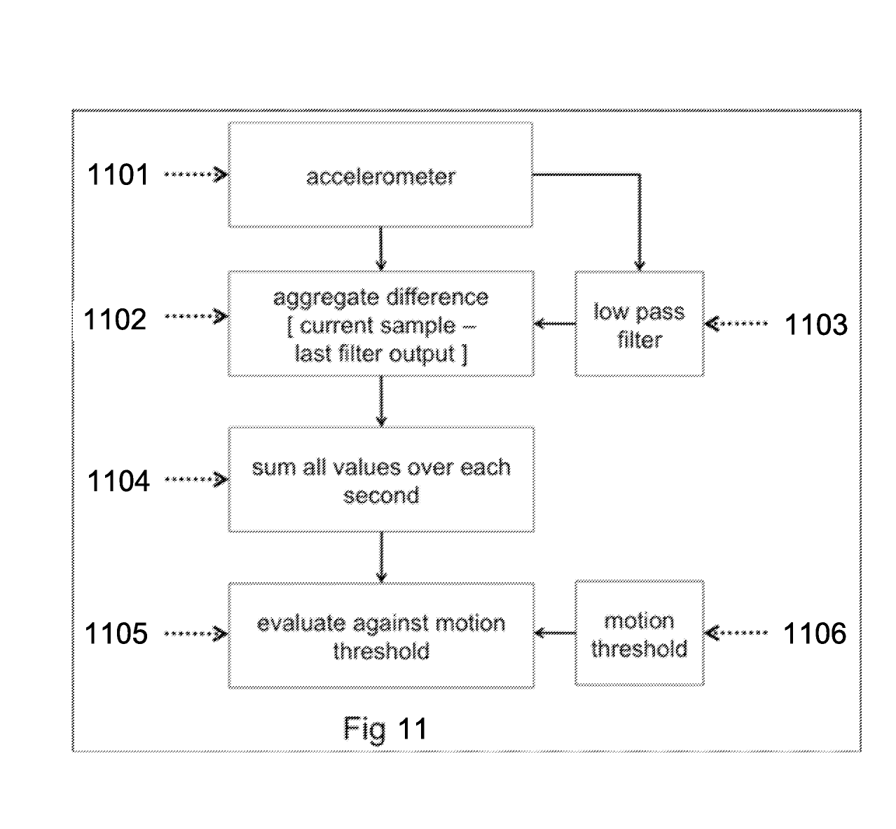

FIG. 11 shows an example of how such motion mitigation may be used. Each sample from an accelerometer 1101 are evaluated 1102 against filtered version of historical values 1103. These differences are summed 1104 over a whole second and evaluated 1105 against a motion threshold 1106 to determine the level of motion contamination for the last second. This level can be used for the determination of whether the data should be used in further processing or not. In this way, a device can evaluate the data being collected via its sensors to determine if sufficient data has been collected (both in quantity and quality) for the evaluation of the user's stress level.

In the case that the processing of data to evaluate a stress parameter is performed on a different processor to that contained in the wearable device, this mechanism still facilitates the ability for the device to alert the wearer to the availability of such data, even if the computation is performed after the recorded data is transmitted for processing remotely.

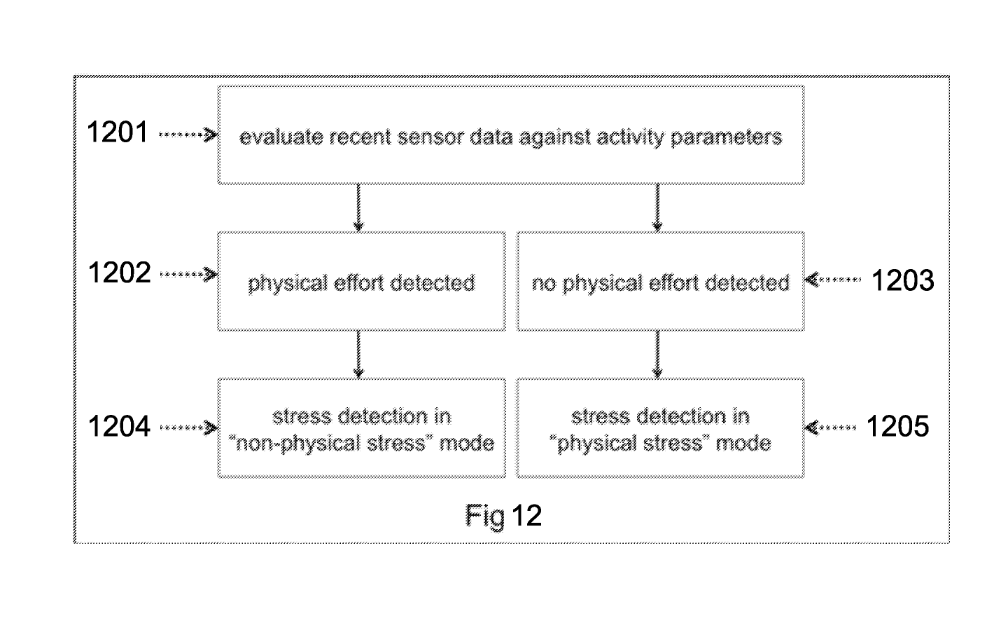

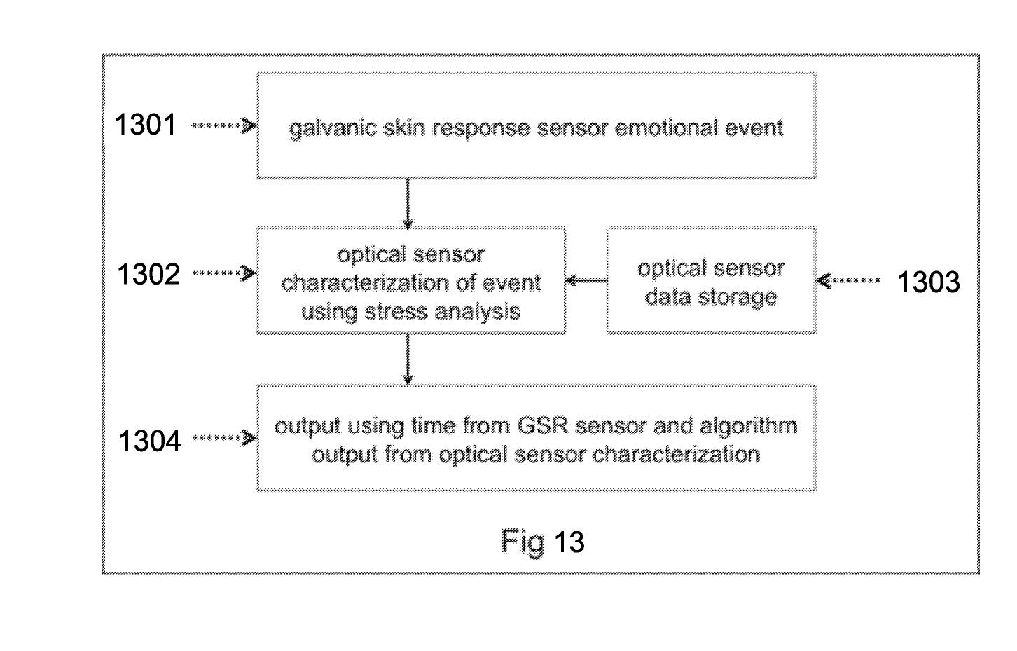

The type, size and nature of the sensed motion may also be used to dynamically select between different feature sets or sensors used in the algorithm, or to add context to the output of the system. FIGS. 12 and 13 illustrate examples of dynamic sensor and feature selection. FIG. 12 illustrates activity parameters, such as the level of motion as detected by an accelerometer, evaluated 1201 against parameters indicating physical effort. Recent data is characterized as being related to physical effort 1202 or not 1203. The subsequent analysis of stress is given a different context for each of non-physical stress 1204 and physical stress 1205. An example of this would be the detection that the user has been on a recent run. The processor 101 would operate in “non-physical stress” mode and discount data that might otherwise be considered as an indication of stress because after these data are consistent with ordinary response to physical exertion. Elevated heart rate, for example, would be associated with the physical effort of the run rather than a stress event.

In some embodiments, when physical exertion is detected the processor 101 does not determine stress. In other embodiments, physical exertion is identified as a type of stress.

FIG. 13 illustrates a second example of how multiple sensors could be used to modify the detection of stress parameters. Skin surface sensors can also be used. Examples include galvanic skin response sensors, perspiration sensors and sweat constituent (cortisol, alcohol, adrenalin, glucose, urea, ammonia, lactate) analysis. In FIG. 13, a galvanic skin response sensor 1301 is used to detect a stress event. Upon detection of a stress event, optical sensor data is analyzed to characterize 1302 the stress at that time. Optical sensor data is saved 1303 in a format allowing analysis of parameters upon detection of a galvanic skin response event. The combined output of this algorithm would be both timing information 1304, as provided by the galvanic skin response event, and characterization as provided by the optical sensor data analysis. In this case the galvanic skin response sensor may also be used as an input to the stress characterization algorithm.

Additional sensors that can be used to provide additional functionality include environmental sensors (e.g., sensors for ultraviolet light, visible light, moisture/humidity, air quality including sensors to detect pollen, dust and other allergens).

### Additional Considerations

The disclosed embodiments beneficially allow for monitoring of a user's stress state over extended periods of time because the device collecting the data is worn by the user and is unobtrusive allowing for the device to be worn continually through most daily activities. Using the additional data collected, the system can determine a context for the individuals and thus provide a more personalized assessment of the user's stress. The more personalized assessment includes providing stress information in relation to the user's own baseline as well as using different methods to determine stress based on the user's activity level.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for identifying and characterizing stress through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1. A device for determining stress of an individual, the device comprising:
an optical emitter to emit light into tissue of a user;
an optical sensor to detect an optical signal from the tissue of the user;
a storage communicatively coupled to the optical sensor to store optical signals;
a processor communicatively coupled to the storage configured to:
retrieve from the storage optical signals detected over a predetermined period of time;
identify features of blood flow in the tissue based on the retrieved optical signal; and
determine a stress state of the user based on the identified features wherein the stress state comprises a type of stress.

2. The device of claim 1 wherein the determined stress state further comprises a level of stress.

3. The device of claim 1 wherein determining a stress state comprises identifying a stress event.

4. The device of claim 1 wherein determining a stress state comprises applying an algorithm to the identified features.

5. The device of claim 1 wherein the algorithm is normalized for the user.

6. The device of claim 1 wherein the optical emitter is configured to emit light having a wavelength between 500 and 600 nanometers (nm).

7. The device of claim 1 wherein the processor is further configured to sample the optical signal at 2 Hz-4096 Hz.

8. The device of claim 7 wherein the processor is configured to sample the optical signal at 20 Hz-1024 Hz, 30 Hz-1000 Hz, 50 Hz-512 Hz, 64 Hz-512 Hz, 100 Hz-256 Hz or 128 Hz-200 Hz.

9. The device of claim 7 wherein the processor is configured to sample the optical signal at 20, 30, 32, 50, 64, 100, 128, 200, 256, 500, 512, 1000 or 1024 Hz.

10. The device of claim 1 wherein the device further comprises a motion sensor configured to identify motion and determining a stress state comprises determining a stress state based on identified motion.

11. A system for determining stress in an individual, the system comprising a processor configured to:
store a plurality of data comprising optical signals of light transmitted through or reflected from tissue of a user;
retrieve data collected during a predetermined period of time;
identify features of blood flow in the tissue based on the retrieved data; and
determine a stress state of the user based on the identified features, the stress state comprising a type of stress.

12. The system of claim 11 wherein one or more features of blood flow are associated with heart rate or heart beat interval.

13. The system of claim 11 wherein determining a stress state comprises applying an algorithm to the identified features.

14. The system of claim 13 wherein the algorithm is normalized for the user.

15. The system of claim 13 wherein the algorithm is normalized based on data collected prior to the predetermined time.

16. The system of claim 11 wherein the processor is further configured to provide for display to the user information associated with the determined stress state.

17. The system of claim 11 wherein the determined stress state further comprises a level of stress.

18. The system of claim 11 wherein determining a stress state comprises identifying a stress event.

19. The system of claim 11 wherein the plurality of data further comprises motion data of the user and determining a stress state is further based on the motion data.

20. The system of claim 11 wherein the transmitted or reflected light has a wavelength of between 500 and 600 nm.
